(19) United States
(12) Patent Application Publication
(10) Pub. No.: US 2011/0159571 A1
(43) Pub. Date: Jun. 30, 2011

(54) ADENOVIRUS TARGETING

(76) Inventors: Michael A. Barry, Rochester, MN (US); Christopher Y. Chen, Rochester, MN (US)

(21) Appl. No.: 12/986,011

(22) Filed: Jan. 6, 2011

Related U.S. Application Data
(63) Continuation-in-part of application No. PCT/US2009/050061, filed on Jul. 9, 2009.
(60) Provisional application No. 61/079,363, filed on Jul. 9, 2008.

(51) Int. Cl. C12N 9/48; C07H 21/00; C07K 14/00; C07K 16/00; C12N 7/00

(52) U.S. Cl. 435/212; 536/23.1; 536/23.53; 530/300; 530/387.3; 435/235.1

(57) ABSTRACT

This document provides methods and materials involved in targeting adenoviruses. For example, this document provides nucleic acid molecules encoding a γ-carboxylated glutamic acid (GLA) domain of a factor X (fX) polypeptide, polypeptides having a GLA domain of a fX polypeptide, adenoviruses containing such nucleic acid molecules, adenoviruses containing such polypeptides, adenoviruses containing such nucleic acid molecules and such polypeptides, and compositions containing therapeutic adenoviral vectors and polypeptides having a GLA domain of an fX polypeptide. In addition, methods and materials for using adenoviruses as viral vectors to deliver nucleic acid to cells other than hepatocytes in vivo, methods and materials for using adenoviruses as vaccines, and methods and materials for using adenoviruses to treat cancer are provided.

### CROSS-REFERENCE TO RELATED APPLICATIONS

This application is a continuation-in-part of International Application Serial No. PCT/US2009/050061, having an international filing date of Jul. 9, 2009, which claims the benefit of U.S. Patent Application Ser. No. 61/079,363, filed Jul. 9, 2008. The disclosure of the prior applications are considered part of (and are incorporated by reference in) the disclosure of this application.

### BACKGROUND

1. Technical Field

This document relates to methods and materials involved in targeting adenoviruses.

For example, this document provides methods and materials for targeting adenoviruses to cells other than hepatocytes in vivo as well as methods and materials for reducing the number of adenoviruses that infect hepatocytes in vivo.

2. Background Information

Adenoviruses are a family of DNA viruses characterized by icosahedral, non-enveloped capsids containing a linear DNA genome. Adenoviruses can be used as viral vectors to deliver nucleic acid to cells, can be used as vaccines, and can be used to treat cancer. When administered to mammals systemically, however, the administered adenoviruses can have a propensity to infect hepatocytes.

### SUMMARY

This document provides methods and materials involved in targeting adenoviruses. For example, this document provides nucleic acid molecules encoding a y-carboxylated glutamic acid (GLA) domain of a factor X (fX) polypeptide, polypeptides having a GLA domain of a fX polypeptide, adenoviruses containing such nucleic acid molecules, adenoviruses containing such polypeptides, adenoviruses containing such nucleic acid molecules and such polypeptides, and compositions containing therapeutic adenoviral vectors and polypeptides having a GLA domain of an fX polypeptide. This document also provides methods and materials for using adenoviruses as viral vectors to deliver nucleic acid to cells other than hepatocytes in vivo, methods and materials for using adenoviruses as vaccines, and methods and materials for using adenoviruses to treat cancer.

As described herein, nucleic acid molecules encoding a GLA domain of an fX polypeptide fused to a ligand binding amino acid sequence (e.g., a single chain antibody) can be used to make a polypeptide that targets adenoviruses to cells expressing the ligand recognized by the ligand binding amino acid sequence. While not being limited to any particular mode of action, the GLA domain of the encoded polypeptide can bind to an hexon polypeptide of an adenovirus, while the ligand binding amino acid sequence of the encoded polypeptide can bind to the ligand present on a particular target cell (e.g., a non-hepatocyte cell), thereby targeting the adenovirus away from hepatocytes and to a desired non-hepatocyte target cell. The nucleic acid molecules provided herein can be designed to lack the ability to encode a functional serine protease domain of an fX polypeptide.

This document is based, in part, on the discovery that polypeptides containing a GLA domain of an fX polypeptide and a ligand binding amino acid sequence (e.g., a single chain antibody) can be used to allow adenoviruses to infect cells expressing the ligand recognized by the ligand binding amino acid sequence. This document also is based, in part, on the discovery that polypeptides containing a GLA domain of an fX polypeptide and a ligand binding amino acid sequence can be used to allow adenoviruses to infect cells not normally infected by adenoviruses. In some cases, a polypeptide containing a GLA domain of an fX polypeptide without the fX polypeptide's native cell binding domain can be used to reduce the ability of adenoviruses to infect liver cells.

In general, one aspect of this document features a nucleic acid molecule comprising, or consisting essentially of, a nucleic acid sequence encoding a polypeptide, wherein the polypeptide comprises, or consisting essentially of, (a) a GLA domain or a GLA variant domain and (b) a ligand binding amino acid sequence. The polypeptide can lack a serine protease domain of a factor X polypeptide. The polypeptide can comprise a human GLA domain of human factor X. The ligand binding amino acid sequence can be a single chain antibody. The single chain antibody can be an anti-Her2, anti-ABCG2, anti-CD19, anti-CD20, or anti-CD38 antibody. The polypeptide can lack the amino acid set forth in SEQ ID NO:9. The polypeptide can comprise an EGF domain of a factor X polypeptide. The polypeptide can comprise a human EGF domain of human factor X.

In another aspect, this document features a polypeptide comprising, or consisting essentially of, (a) a GLA domain or a GLA variant domain and (b) a ligand binding amino acid sequence. The polypeptide can lack a serine protease domain of a factor X polypeptide. The polypeptide can comprise a human GLA domain of human factor X. The ligand binding amino acid sequence can be a single chain antibody. The single chain antibody can be an anti-Her2, anti-ABCG2, anti-CD19, anti-CD20, or anti-CD38 antibody. The polypeptide can lack the amino acid set forth in SEQ ID NO:9. The polypeptide can comprise an EGF domain of a factor X polypeptide. The polypeptide can comprise a human EGF domain of human factor X.

In another aspect, this document features a composition comprising an adenovirus and a polypeptide, wherein the polypeptide comprises (a) a GLA domain or a GLA variant domain and (b) a ligand binding amino acid sequence. The polypeptide can lack a serine protease domain of a factor X polypeptide. The polypeptide can comprise a human GLA domain of human factor X. The ligand binding amino acid sequence can be a single chain antibody. The single chain antibody can be an anti-Her2, anti-ABCG2, anti-CD19, anti-CD20, or anti-CD38 antibody. The polypeptide can lack the amino acid set forth in SEQ ID NO:9. The polypeptide can comprise an EGF domain of a factor X polypeptide. The polypeptide can comprise a human EGF domain of human factor X. The adenovirus can be an anti-cancer adenovirus. The anti-cancer adenovirus can be a GLA-binding oncolytic adenovirus. The adenovirus can be a vaccine adenovirus. The vaccine adenovirus can be an adenovirus expressing influenza hemagglutinin.

In another aspect, this document features a method for targeting an adenovirus to non-liver cells, wherein the method comprises administering, to a mammal, a composition comprising the adenovirus and a polypeptide, wherein the polypeptide comprises (a) a GLA domain or a GLA variant domain and (b) a ligand binding amino acid sequence. The polypeptide can lack a serine protease domain of a factor X polypeptide. The mammal can be a human. The polypeptide can comprise a human GLA domain of human factor X. The ligand binding amino acid sequence can be a single chain antibody. The single chain antibody can be an anti-Her2, anti-ABCG2, anti-CD19, anti-CD20, or anti-CD38 antibody. The polypeptide can lack the amino acid set forth in SEQ ID NO:9. The polypeptide can comprise an EGF domain of a factor X polypeptide. The polypeptide can comprise a human EGF domain of human factor X. The method can comprise mixing the adenovirus and the polypeptide together to make the composition prior to the administration. The method can comprise producing the adenovirus using cells that express the polypeptide under conditions wherein the polypeptide binds to a produced adenovirus, thereby making the composition prior to the administration. The adenovirus can be an anti-cancer adenovirus. The anti-cancer adenovirus can be a GLA-binding oncolytic adenovirus. The adenovirus can be a vaccine adenovirus. The vaccine adenovirus can be an adenovirus expressing influenza hemagglutinin.

In another aspect, this document features a method for targeting an adenovirus to non-liver cells, wherein the method comprises contacting the cells with an adenovirus containing a polypeptide, wherein the polypeptide comprises (a) a GLA domain or a GLA variant domain and (b) a ligand binding amino acid sequence. The polypeptide can lack a serine protease domain of a factor X polypeptide. The polypeptide can comprise a human GLA domain of human factor X. The ligand binding amino acid sequence can be a single chain antibody. The single chain antibody can be an anti-Her2, anti-ABCG2, anti-CD19, anti-CD20, or anti-CD38 antibody. The polypeptide can lack the amino acid set forth in SEQ ID NO:9. The polypeptide can comprise an EGF domain of a factor X polypeptide. The polypeptide can comprise a human EGF domain of human factor X. The adenovirus can be an anti-cancer adenovirus. The anti-cancer adenovirus can be a GLA-binding oncolytic adenovirus. The adenovirus can be a vaccine adenovirus. The vaccine adenovirus can be an adenovirus expressing influenza hemagglutinin. The adenovirus can comprise nucleic acid encoding the polypeptide.

In another aspect, this document features a method for reducing the amount of adenoviruses that infect liver cells, wherein the method comprises administering adenoviruses to a mammal, wherein the adenoviruses contain a polypeptide comprising a GLA domain or a GLA variant domain, wherein the polypeptide lacks a serine protease domain of a factor X polypeptide.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic diagram of the indicated constructs.

FIG. 2 is a graph plotting the percentage of MDA-MB-435 (MDA 435) and SKBr3 cells exhibiting GFP fluorescence following exposure to nothing or supernatants obtained from untransfected 293 cells or 293 cells that were expressing the following fusion protein: GLA-GFP, GLA-EGF-GFP, B1D2-GFP, B1D2-SA-GFP, AAT-B1D2-SA-GFP, GLA-B1D2-GFP, or GLA-EGF-B1D2-GFP. Her 488 is biotinylated Herceptin detected by streptavidin-488 fluorophore, which is a positive control for Her-2 detection. SA488 is streptavidin-488 fluorophore without antibody. MDA-MB-435 cells exhibit low Her-2 polypeptide expression, while SkBr3 cells express high levels of Her-2 polypeptide. The cells were analyzed by flow cytometry for increases in green fluorescence due to binding of a fusion protein containing GFP to the cells.

FIG. 3 (top) is a graph plotting the percentage of untreated and treated MDA-MB-435 (MDA 435), Skov-3 (SKOV3), and SKBr3 cells exhibiting red fluorescence following exposure to Ad Red virus alone or Ad Red virus pre-incubated with supernatants obtained from 293 cells stably transfected to express the indicated construct. FIG. 3 (bottom) is a graph plotting the mean fluorescence index (red fluorescence) for treated and untreated MDA-MB-435 (MDA 435), Skov-3 (SKOV3), and SKBr3 cells following exposure to Ad Red virus alone or Ad Red virus pre-incubated with supernatants obtained from 293 cells stably transfected to express the indicated construct. Skov-3 cells exhibit moderate levels of Her-2 polypeptide. The cells were analyzed by flow cytometry for increases in red fluorescence due to binding and transduction of the cells by the Ad Red virus.

FIG. 4 contains a sequence listing of the nucleic acid sequence (SEQ ID NO:1) of a GLA-B1D2-GFP construct. This construct can express the indicated amino acid sequence (SEQ ID NO:2). Amino acids 1 to 85 (MGRPLHLVLLSASLAGLLLLGESLFIRR-EQANNILARVTRANSFLEEMKKGHLERECMEETCSYEEAREVFEDSDKTNEFWNKY K; SEQ ID NO:3) represent a GLA domain, amino acids 99 to 314 (MPGKGLEYMGLIY-PGDSDTKYSPSFQGQVTISVDKSVSTAYLQWSSLKPSDSAVYFCARHDVGYCTDRTC AKWPEWLGVWGQGTLVTVSSGGGGSGGGGSGGGGSQSVLTQPPSVSAAPGQKVTIS CSGSSSNIGNNYVSWYQQLPGTAPKLLIYDHTNRPAGVPDRFSGSKSGTSASLAISGF RSEDEADYYCASWDYTLSGWVFGGGTKLTVLG; SEQ ID NO:4) represent a B1D1 single chain antibody, and amino acids 320 to 558 (MVSKGEELFTGVVPILVELD-GDVNGHKFSVSGEGEGDATYGKLTLKFICTTGKLPVPWPTLVTTLTYGVQCFSRYPD HMKQHDFFKSAMPEGYVQERTIFFKDDGNYKTRAEVKFEGDTLVNRIELKGIDFKED GNILGHKLEYNYNSHNVYIMADKQKNGIKVNFKIRHNIEDGSVQLADHYQQNTPIGD GPVLLPDNHYLSTQSALSKDPNEKRDHMVLLEFVTAAGITLGMDELYK; SEQ ID NO:5) represent GFP.

FIG. 5 contains a sequence listing of the nucleic acid sequence (SEQ ID NO:6) of a GLA-EGF-B1D2-GFP construct. This construct can express the indicated amino acid sequence (SEQ ID NO:7). Amino acids 1 to 85 (SEQ ID NO:3) represent a GLA domain, amino acids 86 to 123 (DGDQCETSPCQNQGKCKDGLGEYTCTCLEGFEGKNCEL; SEQ ID NO:8) represent an EGF domain, amino acids 137 to 352 (SEQ ID NO:4) represent a B1D1 single chain antibody, and amino acids 358 to 596 (SEQ ID NO:5) represent GFP.

FIG. 6. (A) Secreted GLA-B1D2-GFP and GLA-EGF-B1D2-GFP fusion polypeptides bind to Her-2 positive SKBR3 cells in vitro. Supernatant from 293 cells stably transfected to express various GLA fusion polypeptides or controls was incubated with 1×10⁶ MDA 435 or SKBr3 cells. For Her-2 expression analysis, cells were incubated with biotinylated Herceptin followed by streptavidin 488 incubation. After one hour incubation on ice, cells were washed and analyzed by FACs for green fluorescence. Incubation of adRed virus with GLA-B1D2-GFP or GLA-EGF-B1D2-GFP polypeptide before infection increases transduction of Her-2 positive cells. 1×10⁷ AdRed viral particles were incubated with supernatant from cells expressing GLA-B1D2-GFP, GLA-EGF-B1D2-GFP, or control polypeptides for one hour then applied to cells for 30 minutes at 37° C. Cells were then washed and plated overnight. After 24 hours, cells were trypsinized and analyzed by FACs. (B) Mean Fluorescence Index. (C) Percent dsRed positive. **P<0.01, ***P<0.001.

FIG. 7 is a schematic of GLA fusion polypeptide constructs designed for EGFR and ABCG2 targeting.

FIG. 8. GLA-EGF can be fused to other targeting ScFvs and used to target Ad5. (A) EGFR ScFv fused to GLA-EGF in cell supernatant was used to target Ad-Red to EGFR over-expressing cells lines SKOV3 and MDA-MD-468. Supernatant from 293 cells not secreting the targeting polypeptide is incubated with Ad-Red and used as a negative control. DsRed expression was analyzed after 24 hours by FACS. (B) An ScFv derived from the ABCG2 hybridoma cell line 5D3 was fused to GLA-EGF. The secreted fusion polypeptide was incubated with Ad-Red virus then applied to CHO cells stably transfected to express ABCG2. 24 hours post infection, cells were analyzed for dsRed expression by FACScan.

FIG. 9. Nude mice injected with 3×10⁶ SKOV3 cells i.p. four weeks before treatment were injected with 1×10⁹ Ad-EGFPLuc virus with or without the GLA-EGF targeting fusion polypeptides. (A) Luciferase images were taken at day 3 post-treatment. (B) Quantitation of luciferase expression (mean intensities) from treated mice on day 3 (N=10).

FIG. 10. Tumor transduction in mice. Two mice from each group from FIG. 9 were sacrificed at day 10 or 11 after virus injection and luciferase activity was imaged after the abdominal cavity was opened to facilitate viewing. (A) Mice were imaged immediately after being injected with luciferin and sacrificed. Mouse skin and peritoneum were removed and mouse organs were imaged. (B) Sum intensities of light emitted from organs or tumors were obtained by imaging the tissues after removal from the peritoneum.

FIG. 11. Survival after adenovirus treatment. Mice from FIGS. 9 and 10 were analyzed by Kaplan-Meier survival analysis. Statistical comparisons were performed by log-rank analysis.

FIG. 12 (A). In vitro luciferase expression from SKOV-3 cells infected with Ad-GL. SKOV-3 cells have low CAR expression and are not readily infected by Ad at low MOI. A549 cells infected with either Ad-GLA-αEGFR-RD or Ad-LacZ-RD control virus provided secreted protein via a permeable Transwell. Protein secreted by Ad-GLA-αEGFR-RD-infected A549 cells enhanced the infection of SKOV-3 cells by Ad-GL-RC. n=4. FIG. 12 (B). In vitro cell-killing assay for Ad-GLA-αEGFR-RD virus. SKOV-3 cells plated on a 96-well plate were infected with serially diluted Ad-GLA-αEGFR-RD or control Ad-GL-RC virus. Two weeks after infection, cell viability was assessed by MTT assay (n=4).

FIG. 13. Treatment of SKOV-3 ovarian cancer xenografts with GLA-expressing viruses. Subcutaneous tumors were initiated and treated six times with the indicated virus combinations described in text. Circles represent mice treated with buffer. Triangles represent mice treated with onolytic Ad-GL-RC in combination with replication-defective gene-expressing viruses. Ad-LacZ-RD was the negative control replication-defective virus for Ad-GLA-αEGFR-RD in these groups. Squares represent mice treated with oncolytic Ad-GL-RC in combination with oncolytic replication-competent viruses. Ad-RC was the negative control replication-competent virus for replication-competent Ad-GLA-αEGFR-RD.

Dashed lines represent negative control viruses. Solid lines represent GLA-expressing viruses. (A) Mean tumor volumes. Lines terminate when the first mouse was killed because tumor averages after this point were skewed from the beginning averages. (B) Kaplan-Meier survival analysis through 60 days posttreatment.

### DETAILED DESCRIPTION

This document provides nucleic acid molecules encoding a GLA domain of an fX polypeptide, polypeptides having a GLA domain of an fX polypeptide, adenoviruses containing such nucleic acid molecules, adenoviruses containing such polypeptides, adenoviruses containing such nucleic acid molecules and such polypeptides, and compositions containing therapeutic adenoviral vectors and polypeptides having a GLA domain of an fX polypeptide. This document also provides methods and materials for using adenoviruses as viral vectors to deliver nucleic acid to cells other than hepatocytes in vivo, methods and materials for using adenoviruses as vaccines, and methods and materials for using adenoviruses to treat cancer.

This document provides nucleic acid molecules that encode a GLA domain of an fX polypeptide. The term “nucleic acid” as used herein encompasses both RNA and DNA, including cDNA, genomic DNA, and synthetic (e.g., chemically synthesized) DNA. A nucleic acid can be double-stranded or single-stranded. A single-stranded nucleic acid can be the sense strand or the antisense strand. In addition, a nucleic acid can be circular or linear.

An “isolated nucleic acid” refers to a nucleic acid that is separated from other nucleic acid molecules that are present in a naturally-occurring genome, including nucleic acids that normally flank one or both sides of the nucleic acid in a naturally-occurring genome. The term “isolated” as used herein with respect to nucleic acids also includes any non-naturally-occurring nucleic acid sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., any paramyxovirus, retrovirus, lentivirus, adenovirus, or herpes virus), or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not considered an isolated nucleic acid.

A nucleic acid molecule provided herein can encode a GLA domain of an fX polypeptide (e.g., a human fX polypeptide). A human fX polypeptide can have the amino acid sequence as set forth in GenBank Accession No. NM_{—}000504 (gi no: 4503625). A GLA domain of an fX polypeptide can have the sequence set forth in SEQ ID NO:3. In some cases, a variant GLA domain amino acid sequence can be used as described herein in place of or in addition to a GLA domain. A variant GLA domain amino acid sequence can have an amino acid sequence that is at least 65 percent (e.g., at least 70, 75, 80, 85, 90, 95, or 99 percent) identical to the sequence set forth in SEQ ID NO:3 over that length.

The percent identity between a particular amino acid sequence and the amino acid sequence set forth in SEQ ID NO:3 is determined as follows. First, the amino acid sequences are aligned using the BLAST 2 Sequences (Bl2seq) program from the stand-alone version of BLASTZ containing BLASTP version 2.0.14. This stand-alone version of BLASTZ can be obtained from Fish & Richardson's web site (e.g., www.fr.com/blast/), the U.S. government's National Center for Biotechnology Information web site (www.ncbi.nlm.nih.gov), or the State University of New York-Old Westbury Library (call number: QH 447.M6714). Instructions explaining how to use the Bl2seq program can be found in the readme file accompanying BLASTZ. Bl2seq performs a comparison between two amino acid sequences using the BLASTP algorithm. To compare two amino acid sequences, the options of Bl2seq are set as follows: −i is set to a file containing the first amino acid sequence to be compared (e.g., C:\seq1.txt); −j is set to a file containing the second amino acid sequence to be compared (e.g., C:\seq2.txt); −p is set to blastp; −o is set to any desired file name (e.g., C:\output.txt); and all other options are left at their default setting. For example, the following command can be used to generate an output file containing a comparison between two amino acid sequences: C:\B12seq c:\seq1.txt −j c:\seq2.txt −p blastp −o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical amino acid residue is presented in both sequences. The percent identity is determined by dividing the number of matches by the length of the sequence set forth in SEQ ID NO:3 followed by multiplying the resulting value by 100. For example, an amino acid sequence that has five matches when aligned with the sequence set forth in SEQ ID NO:3 is 94.12 percent identical to the sequence set forth in SEQ ID NO:3 (i.e., 80÷85*100=94.12).

It is noted that the percent identity value is rounded to the nearest tenth. For example, 78.11, 78.12, 78.13, and 78.14 is rounded down to 78.1, while 78.15, 78.16, 78.17, 78.18, and 78.19 is rounded up to 78.2. It also is noted that the length value will always be an integer.

In some cases, a variant GLA domain amino acid sequence can be from 50 to 100 (e.g., 60, 70, 80, 85, 90, 95, or 100) amino acid residues in length and can have the sequence set forth in SEQ ID NO:3 with 15 or less (e.g., 14, 13, 12, 10, 11, 10, 9, 8, 7, 6, 5, 4, 3, 2, 1, or 0) amino acid insertions, deletions, or substitutions. For example, a variant GLA domain amino acid sequence can be 85 amino acid residues in length and can have the sequence set forth in SEQ ID NO:3 with 10 amino acid insertions, deletions, or substitutions. In some cases, a variant GLA domain can contain at least one amino acid substitution relative to the corresponding wild type GLA domain (e.g., human GLA domain of a human fX polypeptide). In some cases, a variant GLA domain can be have the amino acid sequence set forth in SEQ ID NO:3 with five or less (e.g., four or less, three or less, two or less, or one) amino acid insertions, deletions, or substitutions.

Amino acid substitutions can be conservative or non-conservative. Conservative amino acid substitutions replace an amino acid with an amino acid of the same class, whereas non-conservative amino acid substitutions replace an amino acid with an amino acid of a different class. Examples of conservative substitutions include amino acid substitutions within the following groups: (1) glycine and alanine; (2) valine, isoleucine, and leucine; (3) aspartic acid and glutamic acid; (4) asparagine, glutamine, serine, and threonine; (5) lysine, histidine, and arginine; and (6) phenylalanine and tyrosine. Non-conservative amino acid substitutions may replace an amino acid of one class with an amino acid of a different class. Non-conservative substitutions can make a substantial change in the charge or hydrophobicity of the polypeptide product. Non-conservative amino acid substitutions also can make a substantial change in the bulk of the residue side chain, e.g., substituting an alanine residue for an isoleucine residue. Examples of non-conservative substitutions include the substitution of a basic amino acid for a non-polar amino acid or a polar amino acid for an acidic amino acid.

Amino acid insertions, deletions, and substitutions in a nucleic acid molecule can be located at the N-terminus, the C-terminus, or between the N- and C-termini. Nucleic acids encoding a GLA domain of an fX polypeptide can be modified using common molecular cloning techniques (e.g., PCR or site-directed mutagenesis) to generate a variant GLA domain amino acid sequence.

A nucleic acid molecule provided herein can contain a nucleic acid sequence encoding a ligand binding amino acid sequence. In some cases, the nucleic acid sequence encoding a ligand binding amino acid sequence can be designed such that the ligand binding amino acid sequence is expressed as a fusion with a GLA domain of an fX polypeptide. In such cases, the GLA domain can be located at N-terminal to the ligand binding amino acid sequence or C-terminal to the ligand binding amino acid sequence. Examples of ligand binding amino acid sequences include, without limitation, single chain antibodies, peptides, growth factors, and receptor-binding proteins. Examples of ligands recognized by a ligand binding amino acid sequence include, without limitation, Her-2 polypeptides, epidermal growth factor receptors, CD antigens (e.g., CD19, CD20, and CD22), carbohydrates, and ABCG2 transporters.

A nucleic acid molecule provided herein can include additional nucleic acid sequences. Such additional nucleic acid sequences include, without limitation, an EGF domain of an fX polypeptide, marker polypeptides such as GFP, toxins such as cholera toxin, a second antibody sequence, and polypeptides that bind imaging agents. For example, a nucleic acid molecule can encode a fusion polypeptide having a GLA domain, an EGF domain, and a single chain antibody amino acid sequence. In some cases, a nucleic acid molecule provided herein can be incorporated into a viral genome (e.g., an adenoviral genome).

In some cases, a nucleic acid molecule provided herein can be designed to lack the ability to encode a functional serine protease domain of an fX polypeptide. For example, a nucleic acid molecule provided herein can contain a GLA domain of an fX polypeptide and no other portion of an fX polypeptide. The amino acid sequence of a serine protease domain of a human fX polypeptide can be as follows: SVAQATSSSGEAPDSITWKPYDAAD-LDPTENPFDLLDFNQTQPERGDNNLTRIVGGQECKDGECPWQALLINEENEGFCGGTI LSEFYILTAAHCLYQAKRFKVRVGDRNTEQEEGGEAVHEVEVVIKHNRFTKETYDFD IAVLRLKTPITFRMNVAPACLPERDWAESTLMTQKTGIVSGFGRTHEKGRQSTRLKM LEVPYVDRNSCKLSSSFIITQNMFCAGYDTKQEDACQGDSGGPHVTRFKDTYFVTGI VSWGEGCARKGKYGIYTKVTAFLKWIDRSMKTRGLPKAKSHAPEVITSSPLK (SEQ ID NO:9). In some cases, a nucleic acid molecule provided herein can contain a GLA domain of an fX polypeptide and an EGF domain of an fX polypeptide and no other portion of an fX polypeptide.

This document also provides vectors containing a nucleic acid molecule provided herein (e.g., a nucleic acid molecule that encodes GLA domain). Such vectors can be, without limitation, viral vectors, plasmids, phage, and cosmids. For example, vectors can be of viral origin (e.g., vectors derived from adenoviruses, adeno-associated viruses, herpes viruses, lentiviruses, retroviruses, parvoviruses, or Sindbis viruses) or of non-viral origin (e.g., vectors from bacteria or yeast). A nucleic acid provided herein can be inserted into a vector such that a polypeptide containing a GLA domain is expressed. For example, a nucleic acid provided herein can be inserted into an expression vector. “Expression vectors” can contain one or more expression control sequences (e.g., a sequence that controls and regulates the transcription and/or translation of another sequence). Expression control sequences include, without limitation, promoter sequences, transcriptional enhancer elements, and any other nucleic acid elements required for RNA polymerase binding, initiation, or termination of transcription.

Nucleic acid molecules provided herein can be obtained using any appropriate method including, without limitation, common molecular cloning and chemical nucleic acid synthesis techniques. For example, PCR can be used to construct nucleic acid molecules that encode polypeptides having a GLA domain. PCR refers to a procedure or technique in which target nucleic acid is amplified in a manner similar to that described in U.S. Pat. No. 4,683,195, and subsequent modifications of the procedure described therein.

Nucleic acids provided herein can be incorporated into viruses by standard techniques. For example, recombinant techniques can be used to insert a nucleic acid molecule encoding a polypeptide containing a GLA domain into an infective viral cDNA. In some cases, a nucleic acid provided herein can be exogenous to a viral particle, e.g., an expression vector contained within a cell such that the polypeptide encoded by the nucleic acid is expressed by the cell and then incorporated into a new viral particle under conditions where the new viral particle contains the polypeptide and not the nucleic acid.

This document also provides polypeptides encoded by a nucleic acid molecule provided herein. For example, this document provides polypeptides containing a GLA domain of an fX polypeptide. In some cases, a polypeptide provided herein can include a GLA domain of an fX polypeptide and a ligand binding amino acid sequence, while lacking the portion of an fX polypeptide that binds to liver cells. In some cases, a polypeptide provided herein (e.g., a polypeptide containing a GLA domain of an fX polypeptide) can include an additional component linked to the GLA domain. Such additional components can be high binding affinity polypeptides, single chain antibodies, full-length antibodies, drug compounds, or magnetic particles.

Any appropriate method can be used to produce a polypeptide provided herein. For example, a polypeptide provided herein can be produced by standard recombinant technology using heterologous expression vectors. Expression vectors can be introduced into host cells (e.g., by transformation or transfection) for expression of the encoded polypeptide, which then can be purified. Expression systems that can be used for small or large scale production of a polypeptide provided herein include, without limitation, bacterial, yeast, insect, or mammalian cell lines designed to express the polypeptide. Other useful expression systems include insect cell systems infected with recombinant viral expression vectors (e.g., baculovirus) containing a nucleic acid molecule provided herein.

Once produced, a polypeptide provided herein can be purified and/or concentrated. For example, a GLA-EGF-single chain antibody polypeptide can be produced using an heterologous expression system and can be purified using affinity chromatography techniques. In some cases, the purified polypeptide can be concentrated to obtain a polypeptide preparation having a high concentration of purified polypeptide. Such purified and/or concentrated polypeptides can be mixed with adenoviruses as described herein.

This document also provides viruses containing a nucleic acid molecule provided herein, a polypeptide provided herein, or both a nucleic acid molecule and a polypeptide provided herein. For example, this document provides recombinant viruses that contain a nucleic acid that encodes a polypeptide containing a GLA domain.

This document also provides compositions that contain adenoviruses and a polypeptide provided herein. For example, this document provides compositions that contain a mixture of adenoviruses (e.g., therapeutic adenoviruses) and polypeptides containing a GLA domain and a ligand binding amino acid sequence (e.g., a purified and/or concentrated polypeptide). The polypeptide can bind to the adenoviruses via the GLA domain, and the ligand binding amino acid sequence of the polypeptide can direct the adenovirus to infect cells expressing the ligand.

This document also provides methods for targeting adenoviruses to cells. For example, a polypeptide provided herein can be mixed with an adenovirus such that a GLA domain or GLA variant of the polypeptide can bind to the adenoviruses. Then, upon administration to a mammal, a ligand binding amino acid sequence of the polypeptide can interact with the ligand such that the adenovirus infects a cell expressing the ligand. In some cases, a mixture containing adenoviruses and polypeptides containing a GLA domain can be treated with protein cross-linking agents or polyethylene glycol conjugation agents to stabilize these interactions.

In some cases, adenoviruses can be produced using a cell line designed to express a polypeptide provided herein. In such cases, the produced adenoviruses can contain the polypeptide attached via the GLA domain of the polypeptide. In some cases, an adenovirus can be designed to containing the nucleic acid encoding a polypeptide provided herein. In such cases, the expressed polypeptide can bind to the adenoviral particles during virus production in vitro or in vivo.

The invention will be further described in the following examples, which do not limit the scope of the invention described in the claims.

### EXAMPLES

### Example 1

### Targeting Adenoviruses to Cells via Polypeptides Containing a GLA Domain of an fX polypeptide

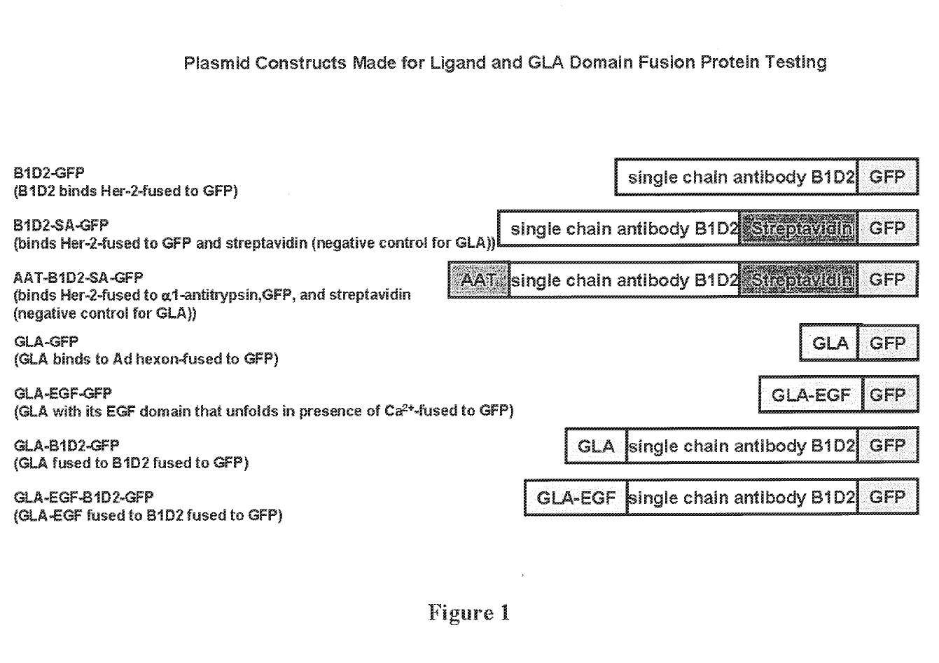

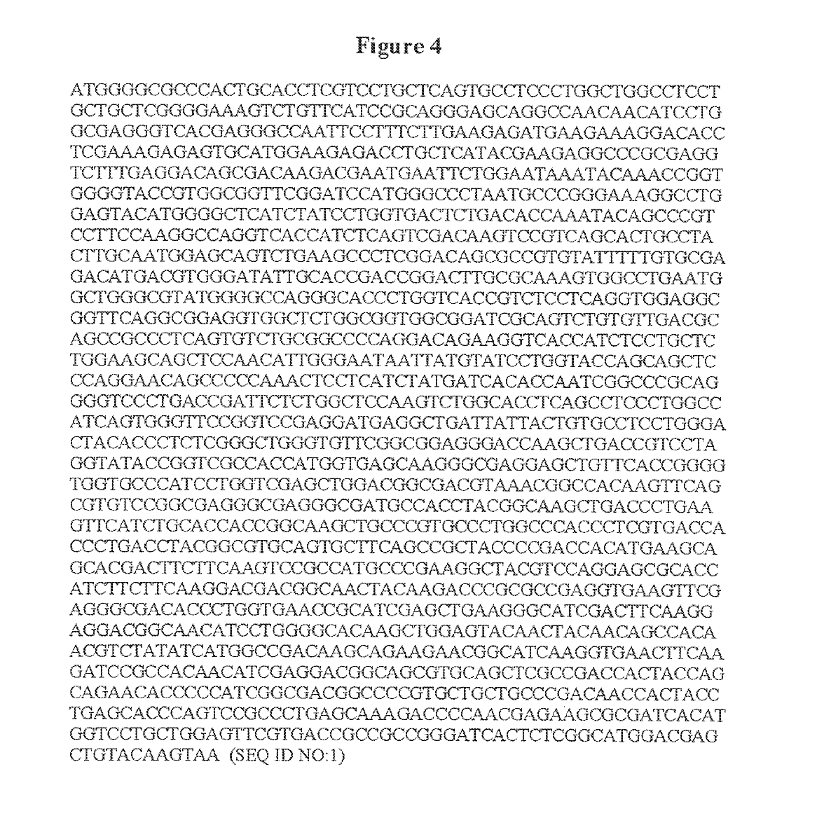

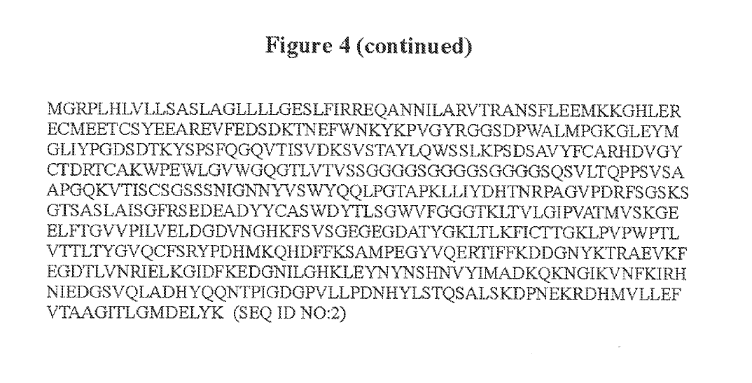

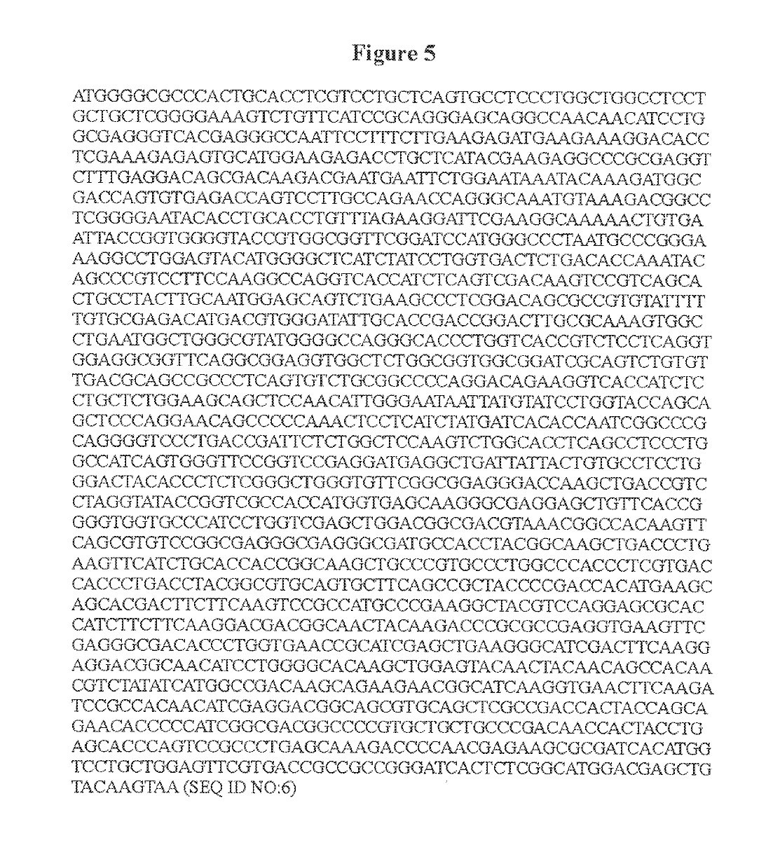

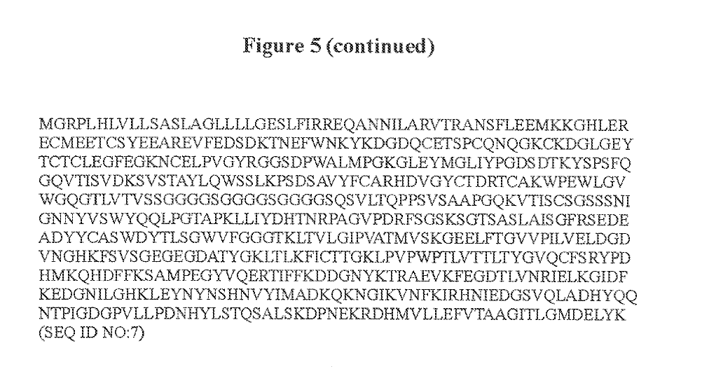

Plasmids expressing various fusion proteins were constructed fusing a GLA domain or GLA-EGF domain plus or minus the B1D2 anti-Her-2 single chain antibody, all of which were fused to GFP (FIG. 1). The B1D2 single chain antibody binds the Her-2 receptor with 1.6×10⁻¹¹ M affinity (Tang et al., J. Immunol., 179:2815-2823 (2007)). Briefly, nucleic acid encoding a GLA domain of an fX polypeptide was fused to nucleic acid encoding a single-chain antibody specific for Her-2 polypeptide and to nucleic acid encoding GFP to create a nucleic acid construct that expresses the GLA domain fused to the single-chain antibody and GFP (FIG. 1). The resulting construct, GLA-B1D2-GFP, had the sequence set forth in FIG. 4. Nucleic acid encoding a GLA domain and EGF domain of an fX polypeptide was also made (FIGS. 1 and 5) as were constructs designed to express (1) a GLA domain fused to GFP, designated GLA-GFP, (2) a GLA domain and EGF domain fused to GFP, designated GLA-EGF-GFP, (3) a B1D2 antibody fused to GFP, designated B1D2-GFP, (4) a B1D2 antibody fused to streptavidin and GFP, designated B1D2-SA-GFP (5) a B1D2 antibody fused to a1-antitrypsin (a protease inhibitor) and streptavidin and GFP, designated AAT-B1D2-SA-GFP, (6) a GLA domain fused to a B1D2 antibody fused to GFP, designated GLA-B1D2-GFP, and (7) a GLA domain and EGF domain fused to a B1D2 antibody fused to GFP, designated GLA-EGF-B1D2-GFP (FIG. 1).

The constructs were tested for the ability to act as a “bridge” to retarget adenoviruses to new receptors. Stable mammalian cell lines were produced using 293 cells, and the supernatants were collected to obtain the secreted fusion polypeptides. These supernatants (1 mL) were incubated for one hour with target cells (SkBr3, Skov-3, and MDA-MB-435 cells). SkBr3 cells exhibit high Her-2 polypeptide expression; Skov-3 cells exhibit medium Her-2 polypeptide expression; and MDA-MB-435 cells exhibit no Her-2 polypeptide expression. These cells were analyzed by flow cytometry for increases in green fluorescence due to binding of the GFP fusion polypeptides to the cells. Her 488 is biotinylated Herceptin detected by streptavidin-488 fluorophore, which is a positive control for Her-2 detection. SA488 is streptavidin-488 fluorophore without antibody.

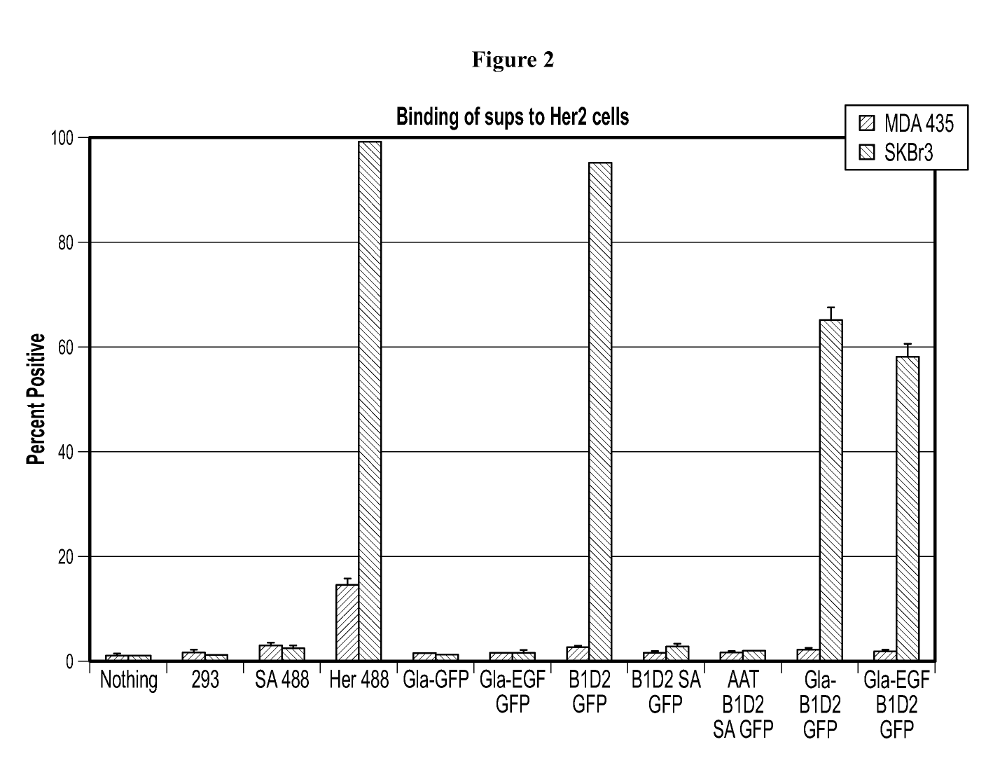

SkBr3 cells incubated with supernatants obtained from cells expressing fusion polypeptides from the B1D2-GFP, GLA-B1D2-GFP, and GLA-EGF-B1D2-GFP constructs exhibited substantial GFP fluorescence, while those incubated with supernatants obtained from cells expressing fusion polypeptides from the GLA-GFP, GLA-EGF-GFP, B1D2-SA-GFP, and ATT-B1D2-SA-GFP constructs did not exhibit substantial GFP fluorescence (FIG. 2). These results demonstrate that GLA domains or GLA-EGF domains can be fused to a ligand binding amino acid sequence without disrupting its ability to bind cells expressing the ligand recognized by the ligand binding amino acid sequence.

In another procedure, the supernatants were incubated with adenoviruses designed to express a red fluorescent polypeptide (dsRed2) for one hour. Then, the viruses were exposed to cells (SkBr3, Skov-3, and MDA-MB-435 cells) for one hour. After 24 hours, the cells were analyzed for gene delivery as evidenced by dsRed expression via flow cytometry.

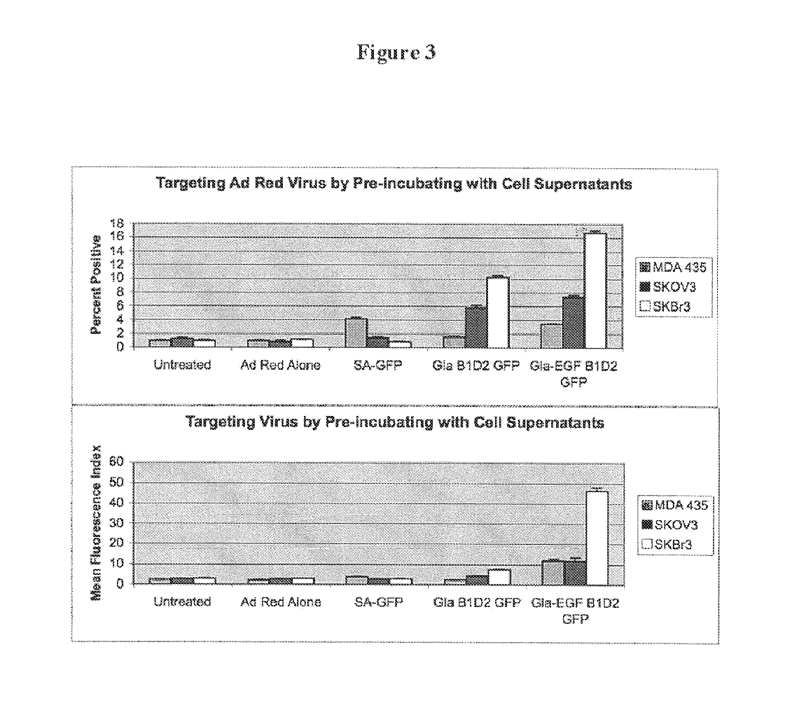

SkBr3 and Skov-3 cells incubated with adenoviruses pre-exposed to supernatants obtained from cells expressing fusion polypeptides from the GLA-B1D2-GFP or GLA-EGF-B1D2-GFP constructs exhibited red fluorescence, while those incubated with adenovirus alone or adenoviruses pre-exposed to supernatants obtained from cells expressing fusion polypeptides from the SA-GFP construct exhibited only background red fluorescence (FIG. 3). These results demonstrate that GLA domains or GLA-EGF domains can be fused to a ligand binding amino acid sequence to create polypeptides having the ability to target adenoviruses to cells expressing the ligand recognized by the ligand binding amino acid sequence (FIG. 3). These results also demonstrate that any appropriate ligand binding amino acid sequence can be used to target adenovirus without disrupting normal adenoviral protein functions.

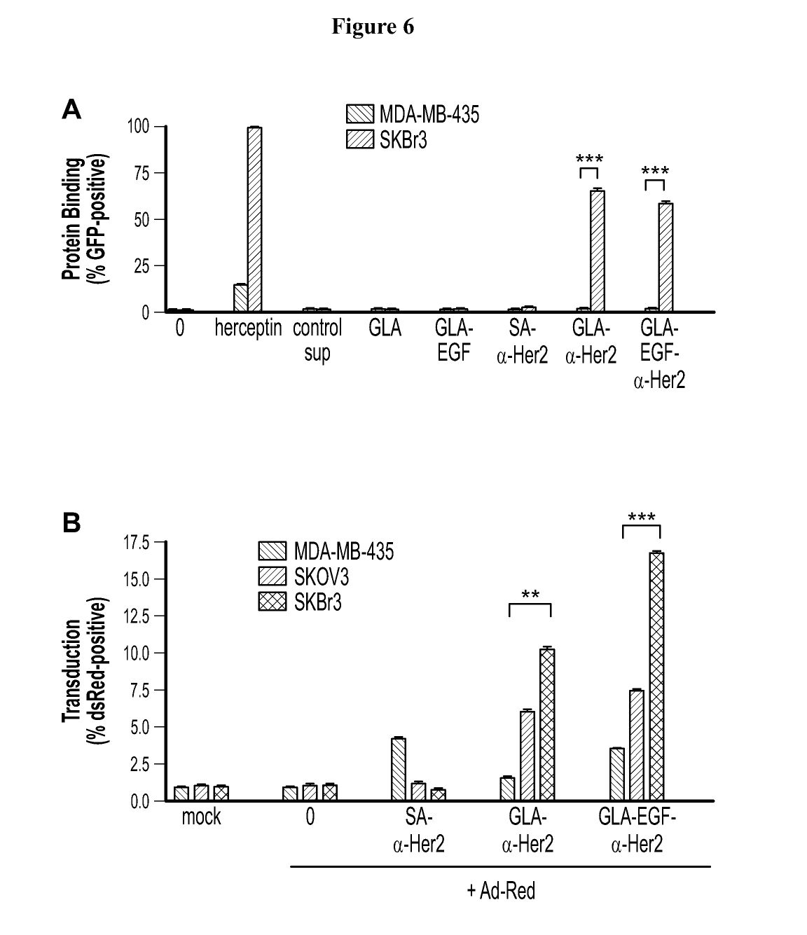

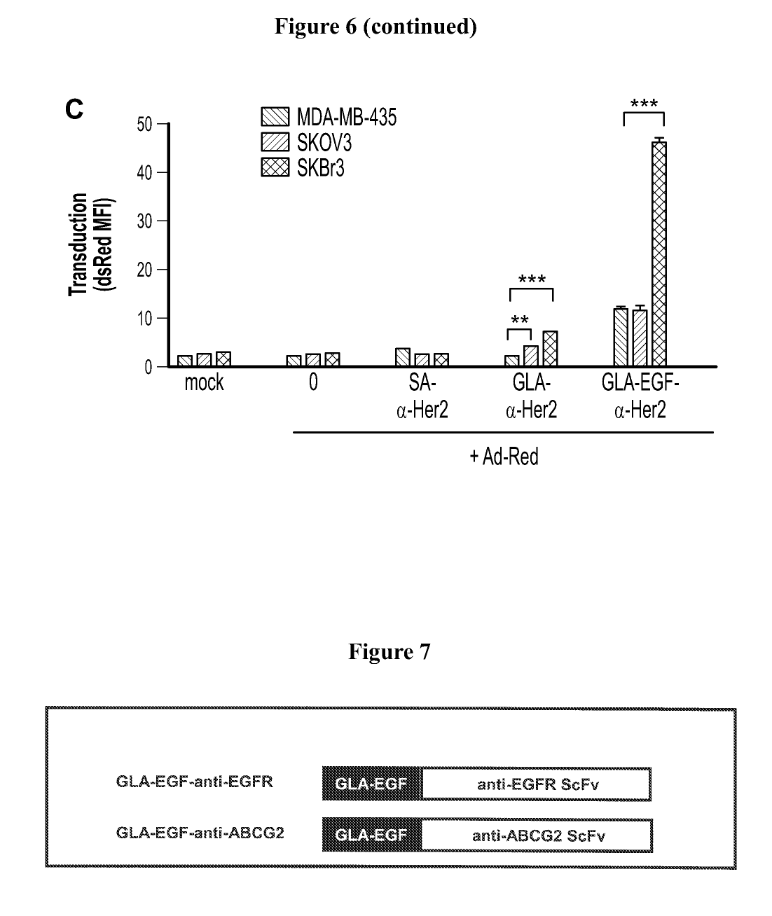

In another study, Ad5 expressing the dsRed2 red fluorescent protein (Ad-Red) was incubated with cell supernatants for one hour. The supernatant with virus was then applied to cells at a multiplicity of infection (MOI) of 10 virus particles (vp) per cell for 30 minutes at 37° C. Targeting was tested on SKBr3 and MDA-MB-435 breast cancer cells along with Her-2-expressing SKOV3 ovarian cancer cells. Cells were washed and were analyzed by flow cytometry for red fluorescence 24 hours later. Both GLA-B1D2-GFP and GLA-EGF-B1D2-GFP fusion polypeptides showed up to 15-fold increased transduction of the Her-2 over non-targeted virus (FIG. 6A). The targeting effect was observed as both an increase in mean fluorescent index (MFI) and the number of dsRed positive cells (FIGS. 6B and 6C). GLA-B1D2-GFP and GLA-EGF-B1D2-GFP both mediated significantly improved transduction to Her-2 positive cells (P<0.01 and P<0.001, respectively). While both fusion polypeptides significantly increased transduction, the GLA-EGF format was markedly more efficient. Therefore, subsequent fusion polypeptides were made with the GLA-EGF polypeptide.

### Example 2

### Targeting Adenoviruses to Cells Expressing EGFR or ABCG2

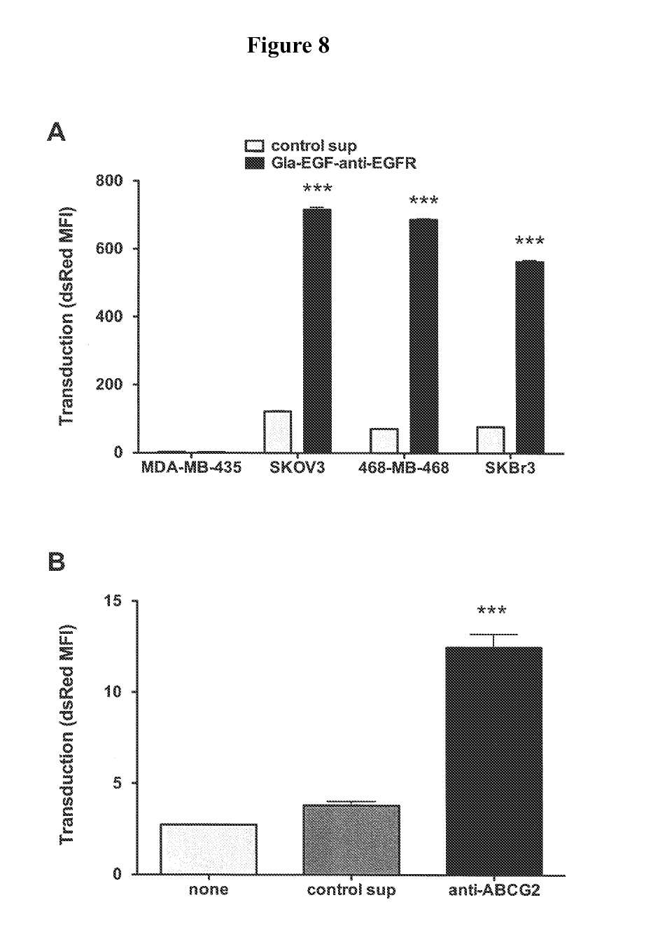

A single chain antibody (ScFv) against EGFR was fused to GLA-EGF without GFP (FIG. 8). Incubation of Ad5 with GLA-EGF-anti-EGFR mediated a 6-fold increase in transduction of EGFR-expressing SKOV-3, SkBr3, and MDA-MB-468 cancer cells when compared to cells treated with virus incubated with control 293 cell supernatants (p<0.001 by ANOVA, FIG. 8A). In contrast, GLA-EGF-anti-EGFR did not enhance transduction of MDA-MB-435 cells, which do not express EGFR.

ABCG2 is an efflux protein that is believed to be responsible for the side-population (SP) phenotype of many adult stem cells and tumor stem cells (Hirschmann-Jax et al., Proc. Nat'l. Acad. Sci. USA, 101:14228-14233 (2004)). In addition to effluxing Hoechst 33342 dye, ABCG2 also effluxes many anti-cancer drugs like mitoxantrone and doxorubicin making these putative stem cells also resistant to chemotherapy. A ScFv against ABCG2 was generated from the anti-ABCG2 hybridoma 5D3. This ScFv was fused to GLA-EGF without GFP (FIG. 7). Pre-incubation of Ad-Red virus with supernatants containing GLA-EGF-anti-ABCG2 before infection of CHO cells stably expressing ABCG2 resulted in greater than 3-fold improvement in dsRed fluorescence when compared to cells infected with virus pre-incubated with 293 supernatant (p<0.001 by ANOVA, FIG. 8B).

### Example 3

### In Vivo Targeting of SKOV3 Tumors using GLA-EGF Fusion Polypeptides

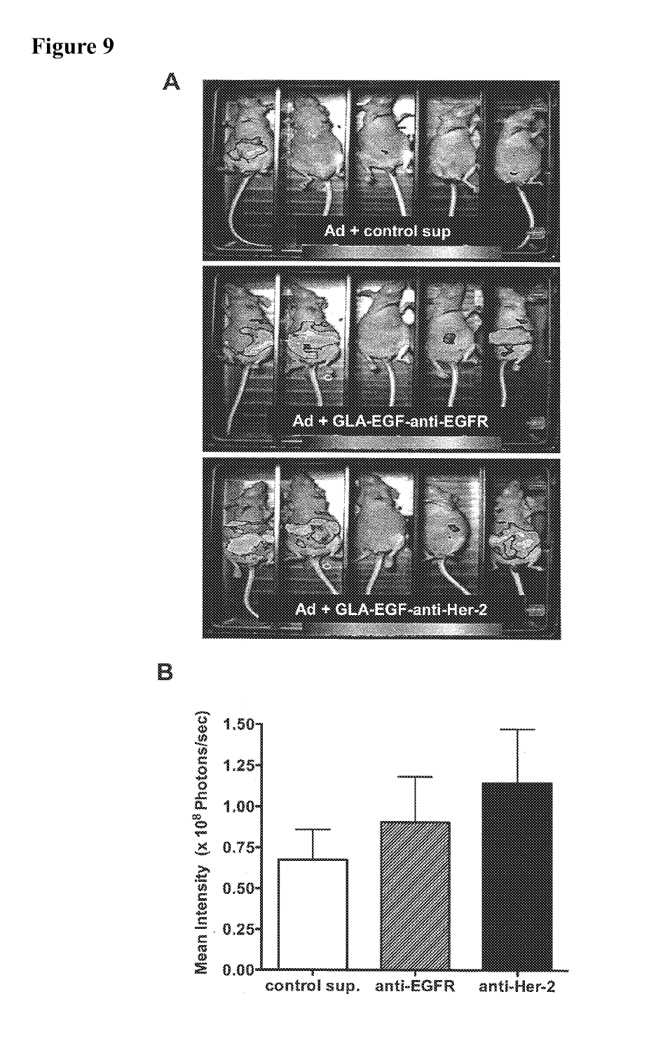

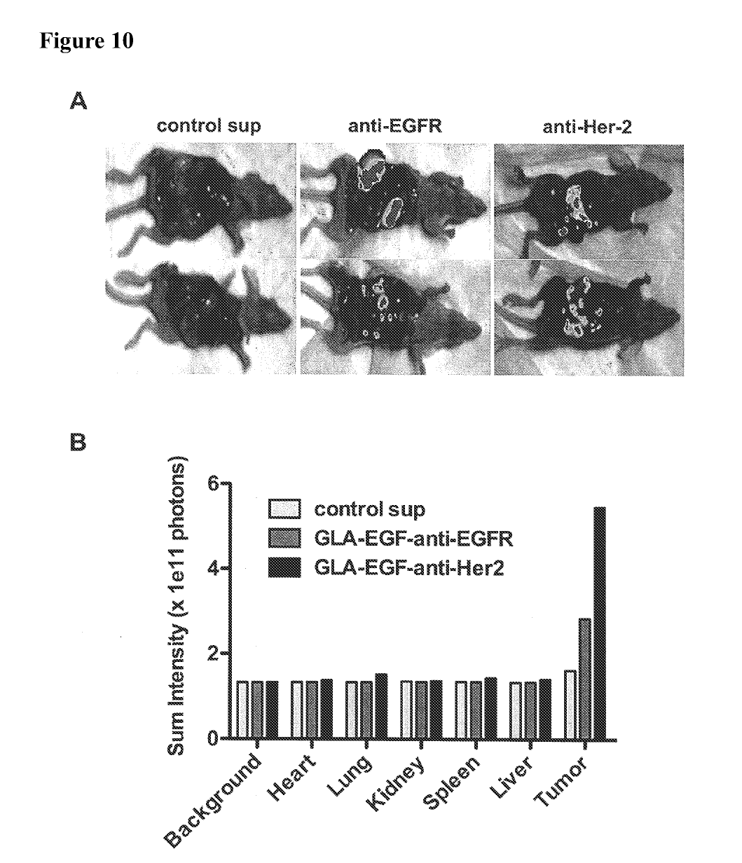

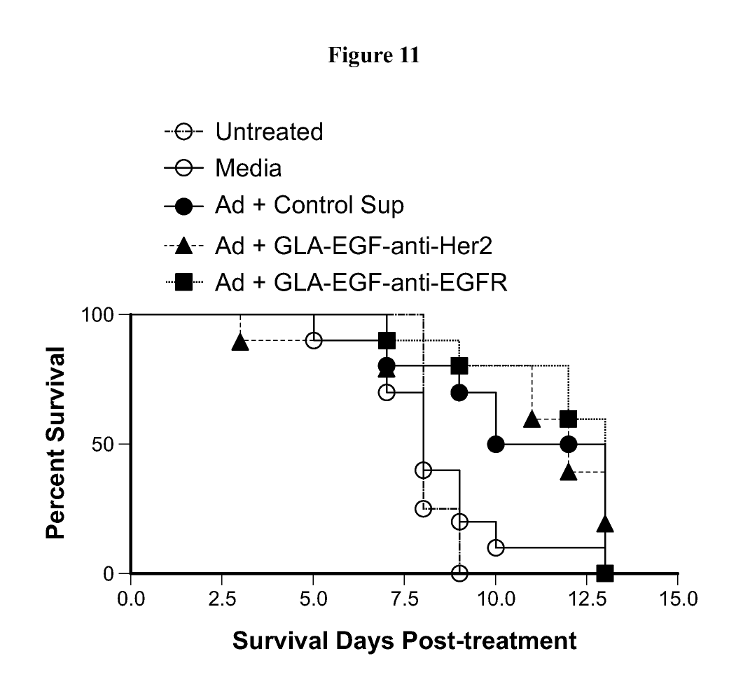

The results provided herein indicate that different ScFvs can be fused to GLA or GLA-EGF to mediate vector retargeting in vitro. To test this in vivo, GLA-EGF-B1D2-GFP and GLA-EGF-anti-EGFR supernatants or 293 supernatants were mixed with replication-competent oncolytic Ad5 expressing EGFP-Luciferase (Ad-EL (Shashkova et al., Cancer Res., 68:5896-5904 (2008)). These virus complexes were injected intraperitoneally into groups of 10 nude mice bearing intraperitoneal (i.p.) SKOV3 tumors, and the mice were imaged 24 hours later for luciferase activity (FIG. 9). Luciferase activity was 30% and 70% higher in mice injected with the anti-EGFR and anti-Her-2 ScFv fusion polypeptides, respectively, than in mice receiving virus with control supernatants (FIGS. 9A and 9B). Due to high variation amongst the mice, these differences did not reach statistical significance (P=0.25 for B1D2 and 0.11 for EGFR targeted tumors). However, animals were sacrificed at day 10 and 11, and peritoneal tumors and organs were imaged. Luciferase activity from the tumors was 3.5 fold higher in the groups that received the fusion polypeptides than in tumors treated with virus and control 293 supernatant (FIG. 10). Transgene expression from ScFv targeted viruses was 2 to 3.5-fold higher in tumors than in other peritoneal organs. In contrast, tumor and organ transduction was comparable in animals receiving 293 supernatants. Survival analysis revealed that animals receiving Ad with 293 supernatants, GLA-EGF-B1D2, and GLA-EGF-anti-EGFR all had significantly improved survival as compared to animals treated with media alone (p=0.030, 0.018, 0.0045, respectively). Median survival for GLA-EGF-B1D2 and anti-EGFR were longer than that of virus treated with control 293 supernatant (FIG. 11). However, these differences in survival were not statistically different (p=0.637 for anti-Her2 and 0.55 for anti-EGFR). These results indicate the GLA-ScFv targeting approach can modify Ad5 tropism both in vitro and in vivo, but that a single round of ScFv targeting may be insufficient to mediate significant improvements in oncolytic activity.

### Example 4

### Methods and Materials

Cell culture. Human cancer cell lines SKOV3 (ovarian carcinoma), SKBr3, MDA-MB-435, and MDA-MB-468 breast carcinoma cells were purchased from the American Type Culture Collection. 293 human embryonic kidney cells were purchased from Microbix. All cell lines were maintained in RPMI 1640 supplemented with 10% fetal bovine serum (FBS; HyClone) and penicillin/streptomycin (HyClone).

Viruses. Ad-Red is a first generation adenovirus that has been rendered replication incompetent due to deletions in E1 and E3. It expresses the dsRed2 transgene from the cytomegalovirus (CMV) promoter. Ad-EGFP-Luc virus is a replication competent virus derived from Ad5 (Shashkova et al., Cancer Res., 68:5896-5904 (2008)). The EGFP-Luciferase fusion gene expresses the enhanced green fluorescent protein fused to firefly luciferase off the CMV promoter. Viruses were purified by double cesium-chloride banding and viral concentrations were determined by A260.

B1D2 fusion protein construct. The B1D2 ScFv was provided by Dr. James D. Marks (University of California San Francisco). B1D2 has sub-nanomolar affinity for the Her-2 receptor (Schier et al., J. Mol. Biol., 255:28-43 (1996)). Human FX gene was purchased from Origene. Primers were designed to PCR either the GLA domain alone (FX 5 prime H3-CTCACTAAGCTTACCATGGGCGCCCACTGCAC (SEQ ID NO:10); FX GLA 3 prime-CTCTGGGATCCGAACCGCCACGGTACCCCACCGGTTTGTAT (SEQ ID NO:11) or the GLA-EGF domains (FX 5 prime H3; FX GLA-EGF 3 prime-CTCTGGGATCCGAACCGCCACGGTACCCCACCGGTAATTCA (SEQ ID NO:12)) from factor X and to insert a 5′ HindIII site and 3′ Age1, Acc65I, and BamHI sites. PCR products were cloned into the HindIII and BamHI sites of B1D2/pEFGP-N1-AAT plasmid. This plasmid is derived from pEGFP and contains a secretion signal peptide followed by the B1D2 single chain antibody (ScFv) fused to GFP. Expression is from the hCMV(IE) promoter. AgeI deletion and re-ligation of the plasmid backbone yielded a control construct with B1D2 deleted. Acc65I-BsrG1 deletion and re-ligation followed by insertion of ligated primers bearing a stop codon (Age-Not stop NC-GGCCGCTTACTAGTCACTCACA (SEQ ID NO:13); Age-Not stop c-CCGGTGTGAGTGATGACTAG (SEQ ID NO:14)) yielded a control construct lacking both the B1D2 and GFP polypeptides. Another control plasmid used was the B1D2/pEGFP-N1-AAT with streptavidin (SA) cloned into the multiple cloning site.

EGFR fusion polypeptide construct. The EGFR ScFv was PCR amplified out of plasmid pTNHaa anti-EGFR provided by Dr. Kah Whye Peng using primers pTNH6-Haa ScFv 5 prime (GGTTCGGATCCATGGGCCCTAATCGAGGGAAGGGCGGCC (SEQ ID NO:15)) and pTNH6-Haa ScFv 3 prime (CTCCACCAATTGGAGTGTACACTAGTGATGGTGATGGTG (SEQ ID NO:16)). The 3′ primer contains a His6 tag for purification purposes. The single-chain was cloned into pCR 2.1 TOPO (Invitrogen) using standard TA cloning methods. EGFR ScFv was then cloned into B1D2/pEGFP-N1-AAT between Apa1 and Kpn1 sites, replacing the B1D2 ScFv.

5D3 fusion protein construct. 5D3, a hybridoma cell line expressing an antibody against the stem cell marker ABCG2, was provided by Dr. Brian Sorrentino (St. Jude Childrens' Cancer Center, Memphis, Tenn.). The 5D3 ScFv was generated using previously published primers and methods. Restriction sites on the amino and carboxy termini of the 5D3 ScFv were added by PCR using primers 5D3 5prime into pEGFP (GGTTCGGATCCATGGGCCCTAG-GCCGAGCTCGATATTCAGATG (SEQ ID NO:17)) and 5D3 3prime into pEGFP (CACATGCGGCCGCTTAGGTGGCGACCGGTATACCTTCCTGGCCGGCCTGGCC (SEQ ID NO:18)). The single chain was TA cloned into pCR 2.1 TOPO. The 5D3 ScFv was then cloned into B1D2/pEGFP-N1-AAT using the restriction sites BamHI and BstZ171 without GFP fusion protein.

Generation and analysis of the GLA and GLA-EGF fusion polypeptides. Fusion polypeptide construct plasmids were stably transfected into 293 cells using Lipofectamine 2000 (Invitrogen) and were selected with 1000 μg/mL G418. Three days post-transfection, cell-free media was collected, and 100 μL aliquots were analyzed for GFP fluorescence using a Beckman-Coulter Multimode DTX 880 plate reader. 30 μL of this media was also run on SDS-PAGE and Western blotted using rabbit polyclonal anti-GFP at 1:2000 dilution and goat anti-rabbit horseradish peroxidase (HRP) at 1:5000 dilution. Visualization of bands was performed using Pierce Femto reagents, and a 5 minute exposure collected by the Kodak In Vivo F system.

Testing fusion polypeptides for binding to Her-2 positive cells. Five mL supernatant from transfected 293 cells expressing the GLA fusion proteins or control proteins were incubated with 10⁶ SKBr3 or MDA-MB-435 cells on ice for 1 hour. Cells were then washed 4 times with 4 mL phosphate buffered saline (PBS) solution then analyzed for green fluorescence using a Becton-Dickinson FACScan. Ten thousand cells were analyzed in each of three replicate tests.

Targeting Ad5 to Her-2 positive cells ABCG2 positive cells or EGFR over-expressing cells. Supernatants from transfected 293 cells expressing the GLA fusion polypeptides or control polypeptides were incubated with purified Ad-Red virus at a concentration of 1×10⁷ viral particles per mL for 1 hour. One mL of supernatant containing virus was applied to 10⁶ SKBr3, SKOV3, or MDA-MB-435, CHO-ABCG2, or CHO cells for 30 minutes at 37° C. Cells were washed four times with 4 mL of PBS and plated onto tissue-culture treated plastic. After 24 hours, cells were removed from plates using cell dissociation buffer (GIBCO). Cells were washed 3 times with PBS and analyzed by flow cytometry on a Becton-Dickinson FACScan for red fluorescence. Ten thousand cells were analyzed in each of three replicate tests.

In vivo targeting of Ad5 to SKOV3 tumor cells using GLA fusion polypeptides. 4-6 week old nude female mice (Harlan) were injected with 4×10⁶ SKOV3 cells by intraperitoneal (i.p) injection on day 0.100 mL of cell supernatant from GLA fusion polypeptides expressing 293 cells or control 293 cells was concentrated using dialysis cassettes 10,000 M.W. cut-off (Pierce) submerged in a dry sucrose bath. After osmotic concentration of the polypeptides, sucrose was dialyzed from concentrated supernatant using three liters of phosphate buffered saline solution (PBS). The concentrated supernatant was incubated with Ad-EGFPLuc replication competent oncolytic adenovirus for two hours at 4 degrees, and unbound polypeptides were removed on centrifugal concentrators. On day 28, late after tumor initiation, mice were injected with 1×10⁹ viral particles of each preparation. Mice survival was monitored, and any mice exhibiting distress or bloating were sacrificed. For imaging studies, mice were anesthetized with isoflurane, then injected with 100 μL of luciferin (20 mg/mL; Molecular Imaging Products) i.p. Mice were imaged with the Roper Lumazone imaging system using 5 minute exposures. For necropsies, mice were injected with luciferin as above immediately before being sacrificed. Images with peritoneal cavity exposed and/or organs were taken with 1 minute exposures. Ten mice were used for each group.

Statistical Analysis. Data were presented as mean value of triplicate measurements unless otherwise noted. Error bars represented the standard deviation. Statistical analysis was performed using PRISM software. Statistical significance was evaluated using one-way ANOVA followed by Bon Ferroni post-test. P<0.05 was considered significant. Survival analysis was performed by log-rank analysis.

### Example 5

### Using a Replication-Defective Adenoviruses Expressing GLA Fusion Protein to Target Oncolytic Ad5 in Trans

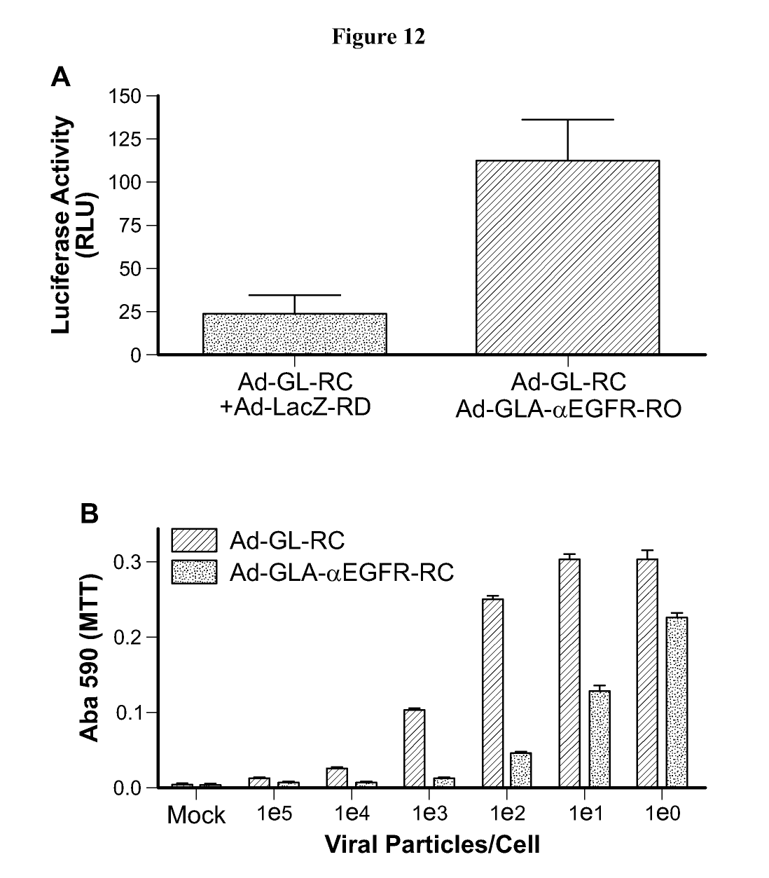

A combination of purified protein with Ad5 could be used for targeting as could the virus expressing its own targeting ligand. To confirm this, a replication-defective E1/E3-deleted Ad5 expressing GLA-αEGFR ScFv (Ad-GLA-αEGFR-RD) was generated. To test the ability of this virus to secrete its ligand and retarget Ad5, it was tested in a paracrine model using Transwell culture plates. Ad5-permissive A549 cells were seeded into the upper wells of a 24-well Transwell plate containing a permeable membrane for cell adhesion. These wells were infected with Ad-GLA-αEGFR-RD or control virus AD-LacZ-RD at an MOI of 1000. Twenty-four hours after infection, the cells were washed to remove free virions before these upper wells were added to Transwell plates with SKOV-3 cells in the bottom wells. Before addition of the upper well, each lower well was infected with Ad-GL-RC virus at an MOI of 10. Under these conditions, untargeted Ad5 would be expected to poorly infect the SKOV-3 cells in the lower wells. If Ad-GLA-αEGFR-RD could provide secreted GLA-αEGFR protein in trans from the upper wells, then SKOV-3 transduction might be increased. Under these conditions, infection of A549 cells by Ad-GLA-αEGFR-RD in the upper wells increased Ad-GL infection of SKOV-3 cells in the lower wells by 470% when compared with Ad-LacZ-RD control virus (FIG. 12A).

### Example 6

### Self-Targeting by Oncolytic Ad5 Expressing a GLA-Targeting Protein In Vitro

One way to use this system to treat tumors would be to have the oncolytic virus express its own GLA fusion protein. This could allow a self-targeting approach in which progeny virus would be targeted by the transgene proteins expressed by the parental viruses. To test this, replication-competent Ad5 expressing GLA-αEGFR (Ad-GLA-αEGFR-RC) was generated by insertion of the CMV-GLA-αEGFR-SV40 cassette into the HpaI site between E1A and E1B in Ad5. This insertion site is the same as was used to generate AD-GL expressing GFP-luciferase. To test its oncolytic ability, oncolytic Ad-GL-RC and AD-GLA-αEGFR-RC were incubated with SKOV-3 cells at various MOIs (FIG. 12B). Determination of cell viability after 14 days by MTT assay revealed that AD-GLA-αEGFR-RC was nearly 100-fold improved in killing of SKOV-3 cells after 14 days.

### Example 7

### In Vivo Oncolytic Activity of GLA-Expressing Viruses

Ad-GLA-αEGFR-RD could retarget itself for gene delivery, but not mediate oncolytic effects because it is replication defective. For oncolytic therapy, AD-GLA-αEGFR-RD could be used in combination with another replication-competent Ad5 to provide targeting ligand in trans as demonstrated in FIG. 12A. This targeting approach would have the safety advantage of separating the targeting moiety from the replication-competent virus, but would require the introduction of two separate viruses. In contrast, Ad-GLA-αEGFR-RC carries its own targeting protein, allowing one virus to be used for therapy.

To perform a direction comparison, Ad-GLA-αEGFR-RD was paired with an oncolytic Ad. Therefore, Ad-GLA-αEGFR-RD was combined with AD-GL-RC to provide oncolysis as well as the ability to image infection with luciferase. The negative control virus group used Ad-LacZ-RD combined with Ad-GL-RC. Ad-GL-RC was also paired with Ad-GLA-αEGFR-RC to remove it as a variable between groups and also to allow for luciferase imaging. The negative control virus for Ad-GLA-αEGFR-RC was Ad-RC lacking any transgene.

The SKOV-3 subcutaneous tumor model was used to allow precise measurement of tumor size that is not feasible in the peritoneal model. Intratumoral virus injections were started 8 days after tumor initiation. One group of eight mice was injected intratumorally with buffer, one group with Ad-GL-RC plus Ad-LacZ-RD, one group with Ad-GL-RC plus AD-GLA-αEGFR-RD, one group with AD-GL-RC plus Ad-RC, and one group with Ad-GL-RC plus AD-GLA-αEGFR-RC. Each group of eight mice received six virus injections over 11 days to mimic a clinical treatment course. A total of 3×10¹° virus particles was injected during each injection, with 1.5×10¹° virus particles of Ad-GL-RC and 1.5×10¹° virus particles of the GLA or control viruses.

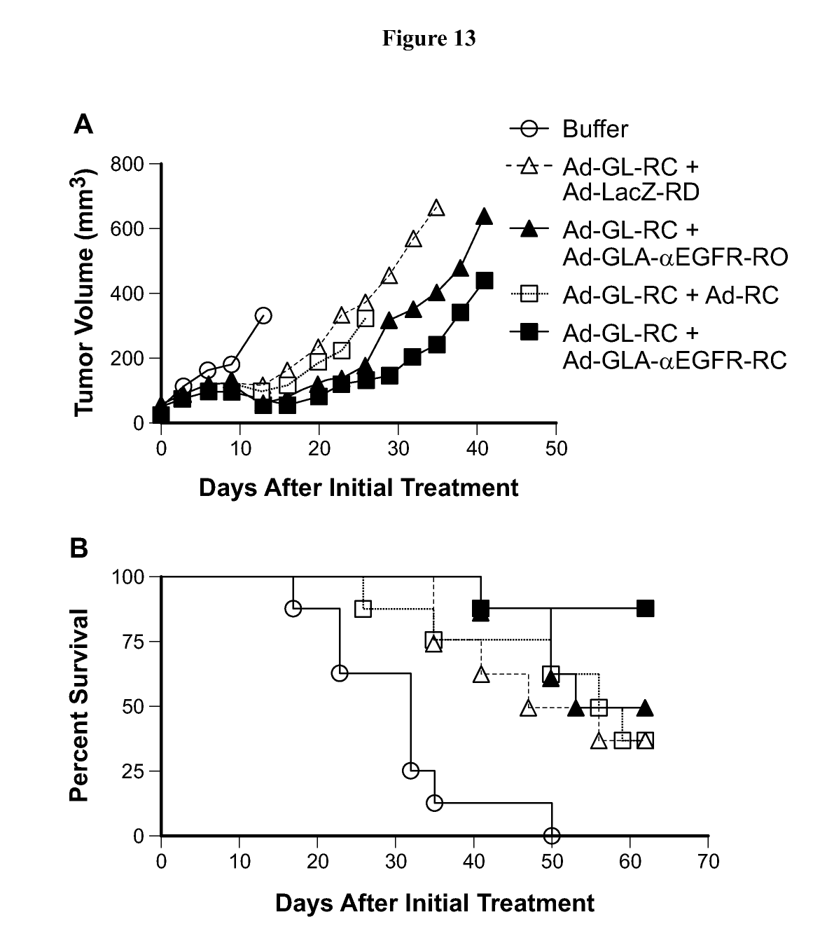

Luciferase imaging after 5 and 11 days of treatment revealed expression due to Ad-GL-RC infection was observed in most of the mice, but was somewhat higher in the AD-GLA-RD group. The tumors grew most rapidly in buffer-treated animals (FIG. 13A), with the first animal having to be killed by day 16 after the start of treatment. Each line in FIG. 13 terminates when the first animal is removed from the group, because the tumor size average no longer applied. Tumor growth was slowed relative to the buffer group for all virus groups, with the greatest delay being observed in the Ad-GLA-αEGFR-RD and Ad-GLA-αEGFR-RC groups. Paired two-tailed t test through day 26 (the last day when all virus groups still retained all mice) demonstrated that the Ad-GLA-αEGFR-RD group had smaller tumor sizes than its control Ad-LacZ-RD group (p=0.0235). Likewise, the Ad-GLA-αEGFR-RC group tumors were smaller than the tumors in its control group with Ad-RC (p=0.0272). These data indicate that expression of GLA protein reduced tumor sizes relative to matched control vectors.

Kaplan-Meier survival curves demonstrated that all of the control mice died by day 50 (FIG. 13B). Death was delay in all of the Ad-treated groups as compared with the buffer group. Fifty to 40% of the mice in the Ad-GLA-αEGFR-RD, Ad-LacZ, and Ad-RC groups survived through day 60, with few differences between the groups. Median survival times were 53, 47, and 56 days for Ad-GLA-αEGFR-RD, Ad-LacZ-RD and Ad-RC, respectively. Therefore, even though Ad-GLA-αEGFR-RD gave smaller tumor sizes than its control Ad-LacZ-RD, this only slightly shifted survival time. This limited response is likely due to the loss of expression of the GLA fusion protein from dying cells. In contrast to the other groups, the Ad-GLA-αEGFR-RC group had significantly better survival with only one of eight animals dying over 60 days (FIG. 13B). Log-rank comparison of the survival curves between Ad-GLA-RC and its control Ad-RC group revealed that the GLA-expressing virus mediated significantly improved survival (p=0.05). These data indicate that an oncolytic adenovirus carrying a GLA targeting protein may mediate better antitumor effects than untargeted Ads.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A nucleic acid molecule comprising a nucleic acid sequence encoding a polypeptide, wherein said polypeptide comprises (a) a GLA domain or a GLA variant domain and (b) a ligand binding amino acid sequence.

2. The nucleic acid of claim 1, wherein said polypeptide lacks a serine protease domain of a factor X polypeptide.

3. The nucleic acid of claim 1, wherein said polypeptide comprises a human GLA domain of human factor X.

4. The nucleic acid of claim 1, wherein said ligand binding amino acid sequence is a single chain antibody.

5. The nucleic acid of claim 4, wherein said single chain antibody is an anti-Her2, anti-ABCG2, anti-EGFR antibody, anti-CD19, anti-CD20, or anti-CD38 antibody.

6. The nucleic acid of claim 4, wherein said single chain antibody is an anti-EGFR antibody.

7. The nucleic acid of claim 1, wherein said polypeptide lacks the amino acid set forth in SEQ ID NO:9.

8. The nucleic acid of claim 1, wherein said polypeptide comprises an EGF domain of a factor X polypeptide.

9. The nucleic acid of claim 1, wherein said polypeptide comprises a human EGF domain of human factor X.

10. A polypeptide comprising (a) a GLA domain or a GLA variant domain and (b) a ligand binding amino acid sequence.

11. The polypeptide of claim 10, wherein said polypeptide lacks a serine protease domain of a factor X polypeptide.

12. The polypeptide of claim 10, wherein said polypeptide comprises a human GLA domain of human factor X.

13. The polypeptide of claim 10, wherein said ligand binding amino acid sequence is a single chain antibody.

14. The polypeptide of claim 13, wherein said single chain antibody is an anti-Her2, anti-ABCG2, anti-CD19, anti-CD20, or anti-CD38 antibody.

15. The polypeptide of claim 13, wherein said single chain antibody is an anti-EGFR antibody.

16. The polypeptide of claim 10, wherein said polypeptide lacks the amino acid set forth in SEQ ID NO:9.

17. The polypeptide of claim 10, wherein said polypeptide comprises an EGF domain of a factor X polypeptide.

18. The polypeptide of claim 10, wherein said polypeptide comprises a human EGF domain of human factor X.

19. A composition comprising an adenovirus and a polypeptide, wherein said polypeptide comprises (a) a GLA domain or a GLA variant domain and (b) a ligand binding amino acid sequence.

20. The composition of claim 19, wherein said polypeptide lacks a serine protease domain of a factor X polypeptide.


SEQUENCE LISTING (18 sequences)

SEQ ID NO: 1 - DNA, 1677 bp, Artificial Sequence
  GLA-B1D2-GFP construct
atggggcgcccactgcacctcgtcctgctcagtgcctccctggctggcctcctgctgctcggggaaagtctgttcatccgcagggagcaggccaacaacatcctggcgagggtcacgagggccaattcctttcttgaagagatgaagaaaggacacctcgaaagagagtgcatggaagagacctgctcatacgaagaggcccgcgaggtctttgaggacagcgacaagacgaatgaattctggaataaatacaaaccggtggggtaccgtggcggttcggatccatgggccctaatgcccgggaaaggcctggagtacatggggctcatctatcctggtgactctgacaccaaatacagcccgtccttccaaggccaggtcaccatctcagtcgacaagtccgtcagcactgcctacttgcaatggagcagtctgaagccctcggacagcgccgtgtatttttgtgcgagacatgacgtgggatattgcaccgaccggacttgcgcaaagtggcctgaatggctgggcgtatggggccagggcaccctggtcaccgtctcctcaggtggaggcggttcaggcggaggtggctctggcggtggcggatcgcagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatctatgatcacaccaatcggcccgcaggggtccctgaccgattctctggctccaagtctggcacctcagcctccctggccatcagtgggttccggtccgaggatgaggctgattattactgtgcctcctgggactacaccctctcgggctgggtgttcggcggagggaccaagctgaccgtcctaggtataccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 2 - protein, 558 aa, Artificial Sequence
  GLA-B1D2-GFP construct
MGRPLHLVLLSASLAGLLLLGESLFIRREQANNILARVTRANSFLEEMKKGHLERECMEETCSYEEAREVFEDSDKTNEFWNKYKPVGYRGGSDPWALMPGKGLEYMGLIYPGDSDTKYSPSFQGQVTISVDKSVSTAYLQWSSLKPSDSAVYFCARHDVGYCTDRTCAKWPEWLGVWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGIPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 3 - protein, 85 aa, Homo sapiens
MGRPLHLVLLSASLAGLLLLGESLFIRREQANNILARVTRANSFLEEMKKGHLERECMEETCSYEEAREVFEDSDKTNEFWNKYK

SEQ ID NO: 4 - protein, 216 aa, Artificial Sequence
  single chain antibody
MPGKGLEYMGLIYPGDSDTKYSPSFQGQVTISVDKSVSTAYLQWSSLKPSDSAVYFCARHDVGYCTDRTCAKWPEWLGVWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLG

SEQ ID NO: 5 - protein, 239 aa, Artificial Sequence
  green fluorescent protein
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 6 - DNA, 1791 bp, Artificial Sequence
  GLA-EGF-B1D2-GFP
atggggcgcccactgcacctcgtcctgctcagtgcctccctggctggcctcctgctgctcggggaaagtctgttcatccgcagggagcaggccaacaacatcctggcgagggtcacgagggccaattcctttcttgaagagatgaagaaaggacacctcgaaagagagtgcatggaagagacctgctcatacgaagaggcccgcgaggtctttgaggacagcgacaagacgaatgaattctggaataaatacaaagatggcgaccagtgtgagaccagtccttgccagaaccagggcaaatgtaaagacggcctcggggaatacacctgcacctgtttagaaggattcgaaggcaaaaactgtgaattaccggtggggtaccgtggcggttcggatccatgggccctaatgcccgggaaaggcctggagtacatggggctcatctatcctggtgactctgacaccaaatacagcccgtccttccaaggccaggtcaccatctcagtcgacaagtccgtcagcactgcctacttgcaatggagcagtctgaagccctcggacagcgccgtgtatttttgtgcgagacatgacgtgggatattgcaccgaccggacttgcgcaaagtggcctgaatggctgggcgtatggggccagggcaccctggtcaccgtctcctcaggtggaggcggttcaggcggaggtggctctggcggtggcggatcgcagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatctatgatcacaccaatcggcccgcaggggtccctgaccgattctctggctccaagtctggcacctcagcctccctggccatcagtgggttccggtccgaggatgaggctgattattactgtgcctcctgggactacaccctctcgggctgggtgttcggcggagggaccaagctgaccgtcctaggtataccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 7 - protein, 596 aa, Artificial Sequence
  GLA-EGF-B1D2-GFP
MGRPLHLVLLSASLAGLLLLGESLFIRREQANNILARVTRANSFLEEMKKGHLERECMEETCSYEEAREVFEDSDKTNEFWNKYKDGDQCETSPCQNQGKCKDGLGEYTCTCLEGFEGKNCELPVGYRGGSDPWALMPGKGLEYMGLIYPGDSDTKYSPSFQGQVTISVDKSVSTAYLQWSSLKPSDSAVYFCARHDVGYCTDRTCAKWPEWLGVWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGIPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 8 - protein, 38 aa, Homo sapiens
DGDQCETSPCQNQGKCKDGLGEYTCTCLEGFEGKNCEL

SEQ ID NO: 9 - protein, 242 aa, Homo sapiens
SVAQATSSSGEAPDSITWKPYDAADLDPTENPFDLLDFNQTQPERGDNNLTRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHV

SEQ ID NO: 10 - DNA, 32 bp, Homo sapiens
ctcactaagcttaccatgggcgcccactgcac

SEQ ID NO: 11 - DNA, 41 bp, Homo sapiens
ctctgggatccgaaccgccacggtaccccaccggtttgtat

SEQ ID NO: 12 - DNA, 41 bp, Homo sapiens
ctctgggatccgaaccgccacggtaccccaccggtaattca

SEQ ID NO: 13 - DNA, 22 bp, Artificial Sequence
  Primer
ggccgcttactagtcactcaca

SEQ ID NO: 14 - DNA, 20 bp, Artificial Sequence
  Primer
ccggtgtgagtgatgactag

SEQ ID NO: 15 - DNA, 39 bp, Artificial Sequence
  Primer
ggttcggatccatgggccctaatcgagggaagggcggcc

SEQ ID NO: 16 - DNA, 39 bp, Artificial Sequence
  Primer
ctccaccaattggagtgtacactagtgatggtgatggtg

SEQ ID NO: 17 - DNA, 43 bp, Artificial Sequence
  Primer
ggttcggatccatgggccctaggccgagctcgatattcagatg

SEQ ID NO: 18 - DNA, 52 bp, Artificial Sequence
  Primer
cacatgcggccgcttaggtggcgaccggtataccttcctggccggcctggcc